SEQUENCE LISTING (3 sequences)

SEQ ID NO: 1 - DNA, 95 bp, synthetic construct
  source 1..95: mol_type = other DNA
caagcagaagacggcatacgagatcgagtaatgtgactggagttcagacgtgtgctcttccgatcaagcagtggtatcaacgcagagtacatggg

SEQ ID NO: 2 - DNA, 95 bp, synthetic construct; identical to SEQ ID NO: 1
  source 1..95: mol_type = other DNA

SEQ ID NO: 3 - DNA, 60 bp, synthetic construct
  source 1..60: mol_type = other DNA
aagcagtggtatcaacgcagagtacnnnyrnnnyrnnnyrnnnattgatggtgcctacag

(19) United States
(12) Patent Application Publication
(10) Pub. No.: US 2024/0327905 A1
(43) Pub. Date: Oct. 3, 2024

(54) ALL-IN-ONE RNA SEQUENCING ASSAY AND USES THEREOF

(71) Applicants: Donald Danforth Plant Science Center, St. Louis, MO (US); The Curators of the University of Missouri, Columbia, MO (US)

(72) Inventors: R. Keith Slotkin, St. Louis, MO (US); Blake Meyers, St. Louis, MO (US); Marianne Kramer, St. Louis, MO (US)

(21) Appl. No.: 18/580,984

(22) PCT Filed: Jul. 20, 2022
(86) PCT No.: PCT/US2022/073956

§ 371 Date: Jan. 19, 2024

Related U.S. Application Data
(60) Provisional application No. 63/223,664, filed on Jul. 20, 2021.

(51) Int. Cl. C12Q 1/6869; C12N 15/10; C12Q 1/6806; C12Q 1/6876

(52) U.S. Cl. CPC C12Q 1/6869; C12N 15/1096; C12Q 1/6806; C12Q 1/6876

(57) ABSTRACT

Provided herein is an all-in-one-RNA sequencing assay that simultaneously delivers a qualitative characterization and quantitative measurement of selected RNAs. Also provided are various uses of the assay.

### CROSS-REFERENCE TO RELATED APPLICATIONS

This application claims priority from Provisional Application No. 63/223,664, filed Jul. 20, 2021, the entire contents of which are hereby incorporated by reference.

### GOVERNMENTAL RIGHTS

This invention was made in part with government support under TI-2016545 awarded by the U.S. National Science Foundation. The government has certain rights in the invention.

### SEQUENCE LISTING

This application contains a Sequence Listing that has been submitted in. XML format via PatentCenter and is hereby incorporated by reference in its entirety. The .XML is named 077875_733716_SequenceListing.xml, and is 4 kilobytes in size.

### FIELD

The present disclosure generally relates to the field of nucleic acid sequencing. More specifically, the present disclosure relates to an all-in-one RNA sequencing assay and various uses thereof.

### BACKGROUND

Transcriptional control of the expression of DNA into RNA is a key regulatory step in nearly every biological process, including the normal development of an organism, dynamic control of a cell's many feedback loops, cell-to-cell signaling and formation of and response to disease. To investigate the RNA molecules that are present in a cell, researchers take advantage of many forms of RNA sequencing (RNA-seq) to obtain a quantitative measurement of the transcript abundance of each RNA, and obtain qualitative information regarding where the transcript begins, ends, how it is spliced and if it contains any mutations. The currently most widely used method of RNA-seq uses sequencing by synthesis (SBS) technology available from Illumina, Inc., which is a short-read sequencing technology that sequences 150 nucleotides at both ends of a fractured mRNA fragment.

In a short-read sequencing method such as the Illumina mRNA-seq method, mRNAs are isolated and fragmented before sequencing into individual reads. Thus, each sequencing read does not represent a full transcript, but rather a small fraction piece of one. This fragmentation, along with the short sequencing reads, creates multi-mapping issues when a single read perfectly matches more than one region of the genome. Second, short reads make it impossible to understand what is happening at the two ends of the same long RNA molecule. For example, the fragments muddle the ability to understand both the exact start and stop site of one individual transcript. Third, genes expressed at very low levels do not obtain many reads, making them difficult to study. Fourth, only mRNAs are examined. The majority of RNA from a locus may however be non-polyadenylated, and any regulation due to these non-polyadenylated reads will be ignored.

Other approaches such as long read sequencing of RNAs (or copied cDNA) use mRNAs that are not fragmented, so each read is a full transcript. This removes issues of multi-mapping and allows full characterization of individual transcripts. However, long read sequencing approaches typically do not analyze any RNAs beyond mRNAs, and the read depth is too low and too over-amplified by PCR to provide a quantitative measure of gene expression.

Against this background, it is also widely recognized that the successful addition of new sequences to a plant genome is highly inefficient, which is a challenge for both academic and industry plant biologists. Industry estimates that ˜1000 transgenic events must be created to identify a mere 1-2 that are sufficiently successful to advance to later, costly commercial development phases. Transgenes that hold promise at early stages may not perform well in later stages of testing, for example due to epigenetic silencing of transcription (i.e., stops RNA and protein production from the transgene). A transgene may make it through early screens, only to be silenced upon becoming homozygous, or when crossed into a different background. Effective, early screening for transgenes that do not perform well is crucial. Discoveries from the field of transgene silencing demonstrate that this process of transcriptional regulation is first triggered on the RNA level, as transgene RNAs fail an unknown internal quality control measure that triggers the transgene to undergo epigenetic silencing. This transgene RNA that fails the quality control process, and triggers transgene silencing, is referred to as “aberrant RNA”, but the exact molecular nature of this type of RNA is not known. Thus, a clear need remains for means to detect all RNAs from a transgene, a gene or a set of genes of interest, and to determine transgene fate.

### SUMMARY

One aspect of the instant disclosure encompasses an all-in-one RNA-sequencing assay. The assay comprises the steps of (1) ligating an RNA, DNA or synthetic adapter to the 3′ end of each RNA molecule among total RNAs or a set of RNAs molecules transcribed from at least one pre-selected locus of an organism's genome, to form ligated RNAs; (2) obtaining full-length cDNA transcripts using the ligated RNAs as input, wherein each cDNA transcript comprises a unique tag identifying each RNA; (3) generating a cDNA sequencing library using the cDNA transcripts, wherein all cDNAs in the sequencing library comprise a multiplex index identifying the library; and (4) sequencing cDNAs of one or more RNA molecules transcribed from the at least one pre-selected locus, thereby obtaining a sequence for each of the RNA molecules from the original RNA sample.

The full-length cDNA transcripts transcribed from the at least one pre-selected locus can be obtained by reverse transcribing the ligated RNAs to obtain full-length cDNA transcripts, wherein each cDNA transcript comprises a unique tag. Sequencing specific cDNAs of transcripts transcribed from the at least one pre-selected locus RNA comprises target capturing specific sequences of interest out of pooled plurality cDNA libraries using oligonucleotide probes to which the cDNA is hybridized, captured, and thereby enriched. In some aspects, the oligonucleotide probes target various endogenous RNAs or exogenous RNAs. The endogenous RNAs can comprise transposable elements, protein-encoding genes, and/or non-coding RNAs.

Sequencing specific cDNAs of RNA molecules of interest can comprise obtaining long reads representing full-length transcripts, thereby providing a long read sequence for each of the RNA molecules that is target captured from the original RNA sample.

The assay can further comprise generating a plurality of cDNA libraries from a plurality of RNA samples, wherein each library comprises cDNAs comprising a multiplex index sequence identifying the library. The RNA samples comprise polyadenylated RNA, non-polyadenylated RNA, partially degraded RNA, partially processed RNA, alternatively spliced variants of RNAs, or transcription start site variants of RNAs. The at least one pre-selected locus comprises a transgene, a gene or a set of genes of interest, a pathogen, or pest sequence within a host organism.

The adapter can be a DNA, RNA, synthetic adaptor, or a combination thereof, that is used to add a cDNA priming site to the 3′ ends of the RNAs. In some aspects, the adapter is the Universal miRNA Cloning Linker.

A DNA oligonucleotide that is complementary to the 3′ adapter can be used as a primer to reverse transcribe the RNAs to the cDNA. The DNA oligonucleotide can comprise the unique tag that is different for each cDNA molecule. The unique tag can be a Unique Molecular Index (UMI) tag. In some aspects, the 3′ adapter is the Universal miRNA Cloning Linker.

The unique tag can allow for distinguishing and collapsing PCR duplicates and enabling quantification of cDNA sequences, and the multiplex index sequence can permit pooling and subsequent demultiplexing of the indexed cDNA libraries.

Another aspect of the instant disclosure encompasses an all-in-one RNA-sequencing assay. The assay comprises the steps of (1) ligating an RNA, DNA or synthetic adapter to the 3′ end of each RNA molecule among total RNAs; (2) reverse transcribing the ligated RNAs to obtain full-length cDNA transcripts, wherein each cDNA transcript comprises a unique tag; (3) generating a plurality of cDNA libraries, wherein each library contains a multiplex index sequence; (4) target capturing specific sequences of interest out of the plurality of cDNA libraries using oligonucleotide probes to which the cDNA is hybridized, captured, and thereby enriched; and (5) sequencing the captured cDNA to obtain long reads representing full-length transcripts, thereby providing a sequence for each of the RNA molecules that is target captured from the original RNA sample. The exogenous RNAs can comprise pest, pathogen, or transgene RNAs.

The cDNA can be captured by biotinylated oligonucleotide probes, and subsequently isolated by magnetic streptavidin beads, washed, and eluted after hybridization. The assay can further comprise amplifying the libraries using primers that do not contain the tags or the multiplex index sequences, permitting amplification of all pool libraries at the same time.

The step of preparing the captured cDNA can comprise end-repairing the cDNA, ligating on adapter sequences, and amplifying the cDNA with primers that comprise the multiplex indexes for multiplexing. The long-read sequencing can comprise Oxford Nanopore-based sequencing of the cDNAs.

The organism can be a plant, animal, fungus, protist, bacterium, archaeon, or virus. In some aspects, the organism is a plant selected from the group consisting of Arabidopsis, corn, soybean, and rice.

An additional aspect of the instant disclosure encompasses a sequencing library of cDNAs each comprising a unique tag generated using an all-in-one assay. The all-in-one assay can be as described herein above. In some aspects, the cDNAs comprise a multiplex index sequence identifying a library/sample.

Yet another aspect of the instant disclosure encompasses a pooled plurality of cDNA libraries generated using an assay of claim 1, wherein each library is generated from an RNA sample, wherein each library comprises the full complement of cDNAs in a sample, wherein each sample comprises a unique tag, and wherein each library comprises a multiplex index.

One aspect of the instant disclosure encompasses a method of detecting or predicting stability of gene expression at a pre-selected locus of an organism's genome. The method comprises sequencing total RNAs or a set of RNAs from the pre-selected locus using an all-in-one RNA-sequencing assay; and processing the long reads to determine gene expression stability. The all-in-one assay can be as described herein above. In some aspects, the processing step comprises demultiplexing the pool into individual libraries; and orienting the long reads to the correct stand of RNA that is present in the organism.

The method can further comprise mapping the reads to the rRNA and tRNA sequences to remove all unwanted or contaminant sequences; mapping the reads that do not map to the rRNA/tRNAs to the target capture sequences; mapping the reads that do not map to the target capture sequences to the entire genome of the organism; and/or calculating the amount of antisense RNA, frequency of 5′ transcript start sites (TSSs), 3′ transcript termination sites (TTSs), splicing pattern, length of poly (A) tail and 3′ polyadenylated sites for the locus.

In some aspects, the method can further comprise determining the features of RNA products, wherein the features comprise the quality and stability of the RNA products determined by metrics selected from the group consisting of amount/percent of RNA that is full-length and polyadenylated, the size of the region where polyadenylation occurs, the amount of sense vs. antisense RNA, the splicing pattern, the fit to periodicity of the known pattern of RNA degradation occurring at the 3′ ends of the exons, and the length of the poly (A) tail. The gene can be transgene and determination of the transgene expression stability leads to prediction of future stability of the expression from the transgene in descendant plants when made homozygous, crossed into different lines, or subjected to post-transcriptional silencing, transcriptional epigenetic silencing or environmental stress.

An additional aspect of the instant disclosure encompasses a method of fast-tracking a stable transgenic event. The method comprises selecting a transgenic event that has the most gene-like transgene expression patterns by using an all-in-one RNA-sequencing assay described herein above. The gene-like transgene expression patterns can comprise accurate transcriptional start sites, patterns of intron splicing, poly (A) tail length and/or clustering of polyadenylation sites.

Another aspect of the instant disclosure encompasses a method of identifying off-type RNAs that trigger RNA decay, RNA degradation, transcriptional or post-transcriptional silencing. The method comprises sequencing total RNAs or a set of RNAs using an all-in-one RNA-sequencing essay described herein above; and processing the long reads to identify off-type RNAs.

Yet another aspect of the instant disclosure encompasses a method of diagnosing a disease in an organism. The method comprises sequencing total RNAs or a set of RNAs from the organism using the all-in-one RNA-sequencing assay of any proceeding claim; and comparing the long reads to one or more reference RNA to identify irregularities in the total RNA or the set of RNAs indicative of the presence of a disease in the organism. In some aspects, the irregularities comprise RNA degradation, RNA instability, incorrect RNA splicing, incorrect RNA processing, alternative transcriptional start or termination sites, shortening of poly (A) tail length and/or RNA decay.

Another aspect of the instant disclosure encompasses a kit for generating cDNA sequencing libraries using an all in one assay described herein above. The kit comprises adapters comprising unique tags, adapters comprising unique indices, primers for generating cDNAs, primers for amplifying libraries, sequencing adapters and primers, or any combination thereof.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

In order to better understand the subject matter that is disclosed herein and to exemplify how it may be carried out in practice, aspects will now be described, by way of non-limiting example only, with reference to the accompanying drawings, in which:

FIG. 1 illustrates production of all-in-one RNA-sequencing libraries, outlining steps to generate cDNA from both polyadenylated and non-polyadenylated RNA, index and target capture and sequence the cDNA.

FIG. 2A illustrates custom adapters and structure of the library molecules that enter into the sequencing step. Color coding is from FIG. 1. The adapters are built from a combination of the NEB Universal miRNA Cloning Linker (red), UMIs from a recent paper (yellow) (Karst et al., Nature Methods, 18 (2): 165-169, 2021), sequences added to perform cDNA synthesis (light green) which are either customized or from the Takara SMARTer cDNA synthesis kit, Illumina sequencing indexes and ends supplied by the NEBNext DNA library kit for Illumina (dark green), which contain indices at the 5′ and 3′ end (blue i7 and red i5), and Oxford Nanopore Technologies (ONT) sequences necessary for long read sequencing (black lines).

FIG. 2B depicts an aspect of a long sequencing read using the assay of the instant disclosure. The color coding matches the color coding in FIG. 2A. The Illumina adapters in Dark Green are provided from the NEBNext Ultra II DNA library preparation kit. The ‘SMART’ sequence in Light Green is provided by the Takara SMARTer cDNA synthesis kit. The NEB Universal miRNA Cloning Linker is in Red. The UMI is in Yellow. i7 and i5 indices are in Blue and Orange.

FIG. 3A illustrates effectiveness of target capture on all-in-one RNA-seq libraries. The qPCR abundance of All-in-One cDNA libraries before and after the target capture step. Upper plot shows qPCR of the libraries before target capture (input), and lower graph shows qPCR of the libraries after target capture. The genes not in the target capture list (gray bars) are reduced to undetectable levels after target capture, while genes on the capture list (blue bars) have increased enrichment. The GUS transgene coding region is not present in this plant, and therefore is not detectable before or after target enrichment.

FIG. 3B. illustrates effectiveness of target capture on all-in-one RNA-seq libraries using a second experiment with different Arabidopsis samples compared to FIG. 3A. The plot on the left is the fraction that did not interact with target capture probes (supernatant), and on the right is the samples that did and are post-enrichment. Blue are regions on the target capture list, and red are not. The genes not on the target capture list are not present post-enrichment, while low-abundance transgene RNAs, such as GUS are not detectable in the supernatant, but accumulate post-enrichment. mPing represents an RNA to which there are probes in the target capture set, but is not present in any samples, thus is not amplified in supernatant or after target capture.

FIG. 3C illustrates effectiveness of target capture on all-in-one RNA-seq libraries using an experiment in Glycine max (soy). The plot on the left is the fraction that did not interact with target capture probes (supernatant), and on the right is the samples that did and are post-enrichment. Similar data to FIG. 3B, targets (red) accumulate to much higher levels after target enrichment, while green are genes not on the target capture list.

FIG. 4 is a flow chart of the custom informatic approach used to demultiplex, orient, quality control and align reads to the regions on the target capture list, illustrating informatic pipeline created to process reads. White boxes represent informatic steps. Light gray boxes are discarded reads, and the blue box represents reads mapped to targets of interest which will be used for data analysis. The green box represents reads that map to the genome but are not targets of interest. These represent any non-specific or background sequences.

FIG. 5 are pie charts illustrating accuracy of the read processing pipeline of the instant disclosure. The GFP reporter gene is only present in one of the samples (RDR6-GFP) that were pooled into one run of the All-in-One RNA-seq method. After sequencing and read processing, the amount of GFP transcripts assigned to each genotype was measured, and found that it was undetectable in the other genotypes, while it accumulated over 7000 reads in the RDR6-GFP genotype. This demonstrates that the read processing bioinformatic approach and execution are occurring accurately.

FIG. 6A illustrates total read coverage for non-polyadenylated reads mapping to a control gene (i.e., Arabidopsis gene AT1G08200). There is an evident enrichment in read coverage in the exons, indicating that this technique captures full-length, spliced RNAs. Evidence of retention of intron 1 in non-polyadenylated reads. It has been previously described that the first intron is the slowest to be spliced out, suggesting that RNAs that are actively being transcribed are identified. Blue tracks are non-polyadenylated read accumulation. The annotation of the gene is on the bottom, with UTRs in orange, exons in blue and introns are white.

FIG. 6B illustrates the location of 5′ ends of all non-polyadenylated reads mapping to a control gene (i.e., Arabidopsis gene AT1G08200). Evidence of strong enrichment of the 5′ end of reads at one site, suggesting the true transcription start site (TSS). Blue tracks are non-polyadenylated read accumulation. The annotation of the gene is on the bottom, with UTRs in orange, exons in blue and introns are white.

FIG. 6C illustrates the location of 3′ ends of all non-polyadenylated reads mapping to a control gene (i.e., Arabidopsis gene AT1G08200). Evidence of enrichment of 3′ end of reads at the 5′ splice site, a feature that has been reported previously in plants, and mammals, demonstrating the reliability and reproducibility of All-in-One RNA-seq. Blue tracks are non-polyadenylated read accumulation. The annotation of the gene is on the bottom, with UTRs in orange, exons in blue and introns are white.

FIG. 6D illustrates total read coverage for polyadenylated reads mapping to a control gene (i.e., Arabidopsis gene AT1G08200), which demonstrates the standard gene-like features of expression. There is an evident enrichment in read coverage in the exons, indicating that this technique captures full-length, spliced polyadenylated RNAs. There is also evidence of build-up of poly (A)+reads in the 3′ end of the gene. Red tracks are polyadenylated reads. The annotation of the gene is on the bottom, with UTRs in orange, exons in blue and introns are white.

FIG. 6E illustrates the location of 5′ ends of all polyadenylated reads mapping to a control gene (i.e., Arabidopsis gene AT1G08200). There is evidence of strong peak in the 5′ end of reads at the TSS, suggesting that some full-length, polyadenylated RNAs are detected. There are many different 5′ ends throughout the 3′ end of the transcript, indicating degradation intermediates from 5′ to 3′ degradation. Red tracks are polyadenylated reads. The annotation of the gene is on the bottom, with UTRs in orange, exons in blue and introns are white.

FIG. 6F illustrates the location of 3′ ends of all polyadenylated reads mapping to a control gene (i.e., Arabidopsis gene AT1G08200). Various 3′ end of reads are observed within the 3′ UTR, suggesting multiple polyadenylation signals, though they are all located within a small region in the 3′ UTR. Red tracks are polyadenylated reads. The annotation of the gene is on the bottom, with UTRs in orange, exons in blue and introns are white.

FIG. 7A illustrates the accumulation of RNAs that are non-polyadenylated and map to the antisense strand of gene targets, wherein percentages of non-polyadenylated reads mapping to the antisense strand are compared between endogenous protein-coding genes, TEs and a transgene are shown. Endogenous protein-coding genes have very few antisense RNAs produced, while TEs have substantially more. Transgenes produce an intermediate amount of antisense RNAs.

FIG. 7B illustrates the percent of RNAs that map to each target that are polyadenylated, wherein percentages of polyadenylated reads mapping to the sense strand are compared between endogenous protein-coding genes, TEs and a transgene are shown. Transgenes have an intermediate percent of polyadenylated RNAs compared to endogenous protein coding genes and TEs.

FIG. 8A illustrates transposable element-like features of expression of non-polyadenylated reads (blue). Non-polyadenylated read accumulation is shown. Red tracks are polyadenylated reads (FIG. 8B). There is as slight build up of reads at the 3′ end is observed (top). No strong 5′ peak which would indicate the TSS for non-poly (A) reads (middle) and 3′ ends of reads are dispersed along the TE (bottom).

FIG. 8B illustrates transposable element-like features of expression of polyadenylated reads (red). Polyadenylated read accumulation is shown. Blue tracks are non-polyadenylated reads (FIG. 8A). There is as slight buildup of reads at the 3′ end is observed (top). No strong 5′ peak which would indicate the TSS for poly (A) reads (middle) and there is one main 3′ end of reads within the TE (bottom).

FIG. 9A illustrates transgene-like features of expression. Non-polyadenylated read accumulation is shown (blue). The annotation of the gene is on the bottom, with exons in blue and introns are white. Peak of 5′ ends of reads at the TSS, and buildup of 3′ ends of reads at the 5′ splice sites is observed, indicating gene-like features. Further, reads mapping to many introns (not just the first intron) and wide-spread 5′ and 3′ ends of reads throughout the entire gene indicate TE-like features.

FIG. 9B illustrate transgene-like features of expression. Polyadenylated reads are shown (red). The annotation of the gene is on the bottom, with exons in blue and introns are white. Various 3′ end of reads within the 3′ UTR and buildup of poly (A)+reads in the 3′ end of the gene indicate gene-like features. The absence of a strong peak in the 5′ end of reads at the TSS indicates TE-like features.

### DETAILED DESCRIPTION

The present disclosure provides, in part, an all-in-one RNA-sequencing assay (“All-In-One RNA-seq”) that avoids limitations of conventional RNA-seq. Assays described herein provide qualitative characterization and quantitative measurement of all RNAs for a select locus or select loci. Full-length RNA transcripts are obtained using long-read sequencing that does not require fragmentation and incorporates molecular indices to quantitatively count reads. The assays provide 10,000-800,000 full-length transcripts per gene, i.e., sequencing depth an order of magnitude beyond the current standard. The assays also provide a new level of resolution for the investigation of RNAs produced from individual loci that are pre-selected by the user. Additional benefits include but are not limited to: increased ability to multiplex many samples at once thus reducing overall cost; enabling the quantification of protein-coding mRNAs, RNA degradation products, and off-type “aberrant” RNAs that may cause gene silencing; and an ability to predict the future stability of gene expression from each locus. The assay can be used to investigate RNAs from any organism, including bacteria, phage, fungi, plants, viruses, and animals, including humans.

### I. Assay

All-In-One RNA-seq assay as disclosed herein comprises ligating an adapter to the ends of all RNAs (cleaved RNAs, mRNAs, non-polyadenylated RNAs, tRNAs, ribosomal RNAs), generating cDNA transcripts of each RNA, and making a sequencing library from all RNAs. To obtain the sequencing depth desired from the selected genes, a target-capture step can be applied to select a number of loci from the genome to study in detail. Any number of loci can be selected to study in detail. For instance, 1, 5, 10 15, 20, 25, 30, 35, 40, 45, 50, 55, 60, 65, 70, 75, 80, 85, 90, 95, 100 or more, 1,000 or more, or 10,000 or more loci can be selected.

Selected regions can then be subjected to sequencing of their cDNAs. In some aspects, sequencing can be long read sequencing using methods known to individuals of skill in the art. Non-limiting examples of long read sequencing methods include Oxford Nanopore Technology (ONT) sequencing (Karst et al., Nature Methods, 18 (2): 165-169, 2021) and PacBio Iso-Seq sequencing. In some aspects, long read sequencing comprises Oxford Nanopore Technologies sequencing. In other aspects, long read sequencing comprises PacBio Iso-Seq sequencing. Other optional elements in the assays described herein include unique molecular indices to measure expression quantitatively, and more multiplex indices to sequence many libraries at once thus reducing overall assay cost.

On aspect of the present disclosure provides an all-in-one RNA sequencing assay (“All-In-One RNA-seq”) that offers a qualitative characterization and quantitative measurement of all RNAs for a select locus or select loci. The assay of the instant disclosure can be used to characterize all forms of RNAs including, without limitation, polyadenylated, non-polyadenylated, partially degraded RNA, partially processed RNA, alternatively spliced variants, and transcription start site variants of RNAs, tRNAs, ribosomal RNAs, among others. The assay can provide quantitative measurements of all variants of an RNA transcribed from a select locus or select loci, as well as post-transcriptionally processed variants of the RNA transcribed from the select locus or select loci.

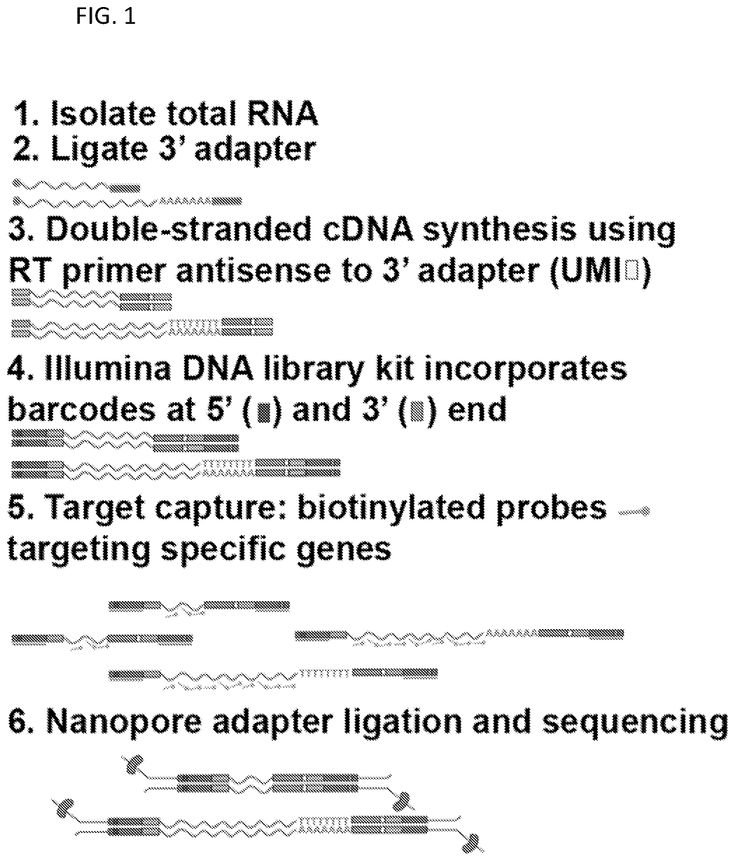

An All-In-One RNA-seq assay of the instant disclosure first comprises ligating a polynucleotide adapter to the 3′ end of each RNA molecule among total RNAs to form ligated RNAs. The adapter is ligated to all forms of RNAs described herein, thereby capturing all forms of the RNA for analysis using the All-In-One RNA-seq assay. Any nucleic acid sequence may be used as an adapter provided it can be ligated to the 3′ end of RNA. The adapter may be an RNA, DNA or synthetic adapter, or a combination thereof. The adapter can also be or comprises modified nucleic acid bases, such as modified DNA bases or modified RNA bases. Modifications may occur at, but are not restricted to, the sugar 2′ position, the C-5 position of pyrimidines, and the 8-position of purines. Examples of suitable modified DNA or RNA bases include 2′-fluoro nucleotides, 2′-amino nucleotides, 5′-aminoallyl-2′-fluoro nucleotides and phosphorothioate nucleotides (monothiophosphate and dithiophosphate). The adapter can also be or comprise nucleotide mimics. Examples of nucleotide mimics include locked nucleic acids (LNA), peptide nucleic acids (PNA), and phosphorodiamidate morpholine oligomers (PMO). By way of non-limiting example only, the adapter may be the commercially available Universal miRNA Cloning Linker (New England Biosciences) (Step 2 in FIG. 1).

As described above, RNA sequencing methods currently widely used such as Illumina mRNA-seq, examines only mRNAs, while the majority of RNA from a locus may be non-polyadenylated, which means any regulation due to these non-polyadenylated reads is ignored by RNA-seq. This drawback is alleviated by All-In-One RNA-seq because it examines all RNA forms present in a cell. Non-limiting examples of RNA forms include protein-coding messenger RNAs (mRNAs), or non-coding RNAs. Non-coding RNAs (“ncRNA”) can be encoded by their own genes (RNA genes) but can also derive from protein-coding genes or mRNA introns. Non-limiting examples of non-coding RNAs include transfer RNA (tRNA), ribosomal RNA (rRNA), miRNAs, long non-coding RNAs (lncRNA), long non-translated RNAs (IntRNA), trans-acting siRNAs (tasiRNAs), antisense mRNAs, enhancer RNAs, introns, snRNAs, snoRNAs, and ribozymes. RNA molecules can also be viral genomes, transposable elements, and viral transcripts. RNA forms can also include polyadenylated RNAs, non-polyadenylated RNAs, precursor RNAs, partially degraded, partially processed, alternatively spliced variants of RNAs, and transcription start site variants of RNAs, among others.

The RNAs can be encoded by endogenous genes or exogenously introduced genes. In some aspects, the at least one pre-selected locus may comprise a transgene, a gene, or a set of genes of interest, a pathogen or pest sequence within a host organism. For instance, in the case of a pathogen or pest sequence within a host organism, one can isolate the malaria RNAs, for instance, out of an infected person to study the infection using the all-in-one RNA-sequencing assay disclosed above and herein. In the case of the transgene, analysis of the total RNAs or set of RNAs from the select locus (loci) could predict stability of the transgene expression, thus expedite identification of a stable transgenic event.

Next, All-In-One RNA-seq comprises generating full-length cDNA transcripts of each ligated RNA. Methods of generating full-length cDNA transcripts are known in the art and can comprise reverse transcribing RNAs. Currently used methods of generating full length cDNAs for long form sequences comprises using an oligo-dT primer for reverse transcriptase that binds the polyadenylated tail of mRNAs. This is a limitation of conventional RNA-seq because only polyadenylated RNAs are captured, missing non-polyadenylated RNAs and RNAs that have been already deadenylated. Conversely, an assay of the instant disclosure comprises use of a reverse transcriptase primer that binds the 3′ adapter in the ligated RNAs to reverse transcribe the RNAs to the cDNA. This way, all ligated RNAs, including non-polyadenylated can be reverse transcribed (Step 3 in FIG. 1).

In an assay of the instant disclosure, each full-length cDNA transcript comprises a unique tag. These tags can be added before any PCR amplification steps, thus enabling the accurate identification of PCR duplicates. Unique tags, design, and methods of synthesis of unique tags are known to individuals of skill in the art. Unique tag sequences can comprise from about 4 to about 10 or more nucleotides. In some aspects, the length of a unique tag sequence can be about 4, 5, 6, 7, 8, 9, 10, 15, 20, 25, 30, 35, 40, 45, 50, 55, or 60 nucleotides, or longer. In some aspects, the length of a unique tag sequence can be a multiple repeating pattern of at least 15, at least about 18 nucleotides, or longer. In some aspects, the length of a unique tag sequence can be a 15× (ONT R10.3), 25× (ONT R9.4.1), 3× (Pacific Biosciences circular consensus sequencing), or a combination thereof (Karst et al., Nature Methods, 18 (2): 165-169, 2021).

In some aspects, the unique tag is a Unique Molecular Identifier (UMI) tag (Step 3 in FIG. 1). UMIs, also known as “Molecular Barcodes”, are short sequences or molecular tags that are added to DNA or RNA fragments in some long-read sequencing library preparation protocols to identify the individual input DNA or RNA molecule in a population of DNA or RNA molecules. UMIs can be used to reduce errors and quantitative bias introduced by the amplification. Suitable UMIs for All-In-One RNA-seq can be those used for Oxford Nanopore Technologies sequencing (Karst et al., Nature Methods, 18 (2): 165-169, 2021).

Methods of adding a UMI to one or a plurality of DNA molecules is known in the art and include ligating a nucleic acid sequence comprising the UMI to each cDNA molecule, or using a primer comprising a UMI for reverse transcription of RNA molecules to cDNAs, each of which comprises a UMI. In an aspect, an assay of the instant disclosure comprises introducing the UMI by reverse transcribing the ligated RNAs using an oligonucleotide that is complementary to the 3′ adapter of the ligated RNAs, wherein each oligonucleotide comprises a UMI to obtain the full-length cDNA transcripts, each of which comprises a UMI. The UMI is unique for each cDNA molecule. Since there is a 1:1 relationship between cDNA and RNA (cDNA is copied directly from the RNA), the UMI is also unique for each RNA molecule. The UMI allows for distinguishing and collapsing PCR duplicates and enabling quantification of cDNA sequences into the number of original RNA samples present in the sample.

The double stranded cDNAs generated above are next used as input for generating a sequencing library, wherein all cDNAs in the sequencing library comprise a unique multiplex index identifying the library (Step 4 in FIG. 1). In some aspects, all cDNAs in the sequencing library comprise an index sequence identifying the library. At this step, a cDNA library possesses two essential features: 1) a UMI distinguishing PCR duplicates and enabling quantification of long-read RNA-seq and 2) a multiplex index sequence identifying a library/sample. To facilitate subsequent manipulation, a cDNA library can be amplified using primers that do not comprise UMIs, thus permitting amplification of all cDNAs at the same time.

A plurality of cDNA libraries, each generated from RNA samples for sequencing can be generated, wherein each library comprises the full complement of cDNAs in a sample, wherein each sample comprises a unique tag, and wherein each library comprises a unique index. In some aspects, the library is an Illumina DNA sequencing library prepared using the NEBNext DNA library kit for Illumina. This kit first end-repairs the cDNA molecules before ligating DNA adapter sequences to the ends of each cDNA molecule to allow for a subsequent PCR to add standard 8-nucleotide Illumina indices unique for each library/sample.

The plurality of cDNA libraries can be pooled for multiplex sequencing and then subsequent demultiplexing using the index sequences, thereby making this protocol extremely scalable. That is, the index sequence contained in each cDNA library permits pooling and subsequent demultiplexing of the indexed cDNA libraries. In some aspects, the pooled libraries are amplified using primers that do not contain the tag unique to each cDNA or the index sequences unique to each library, thus permitting amplification of all pool libraries at the same time.

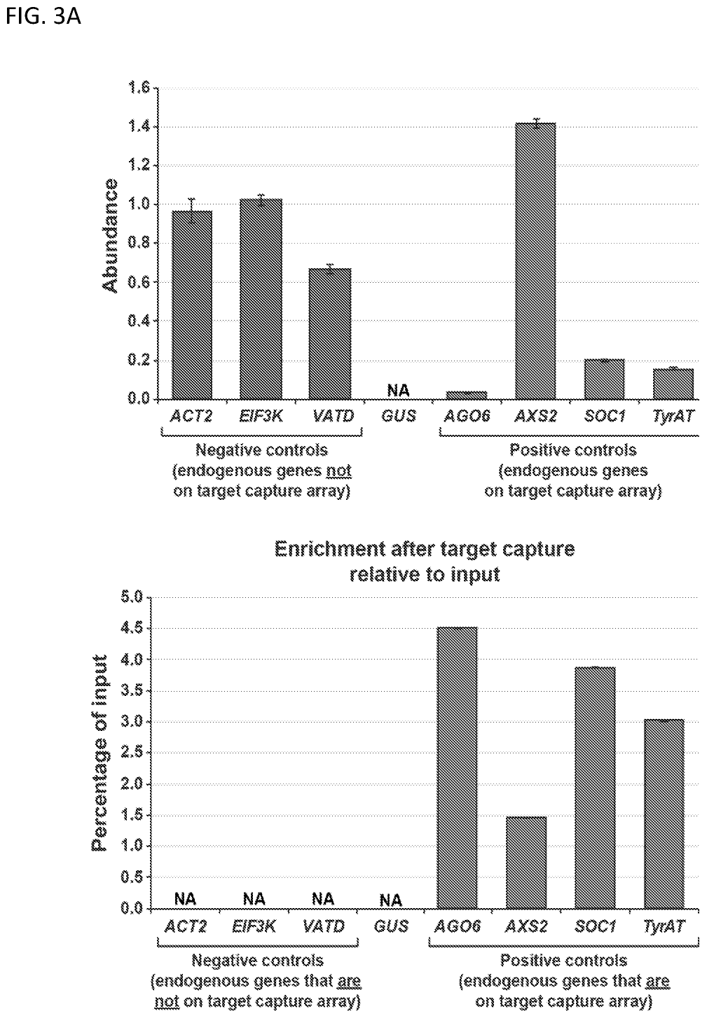

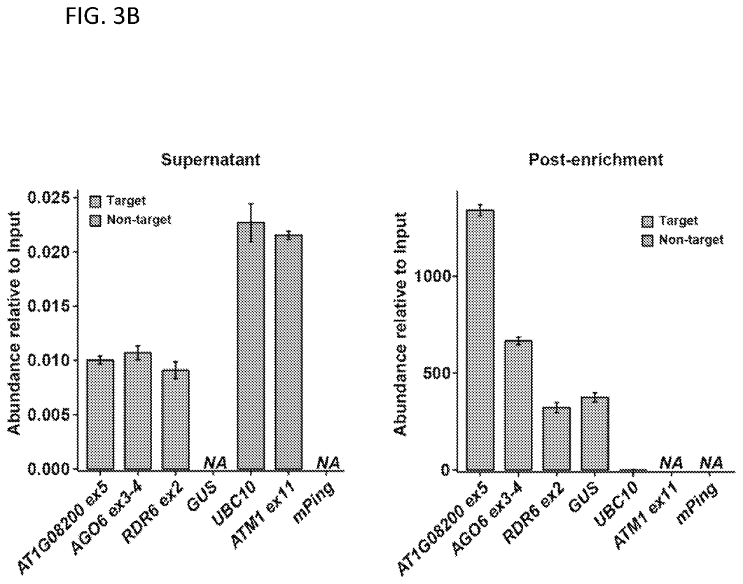

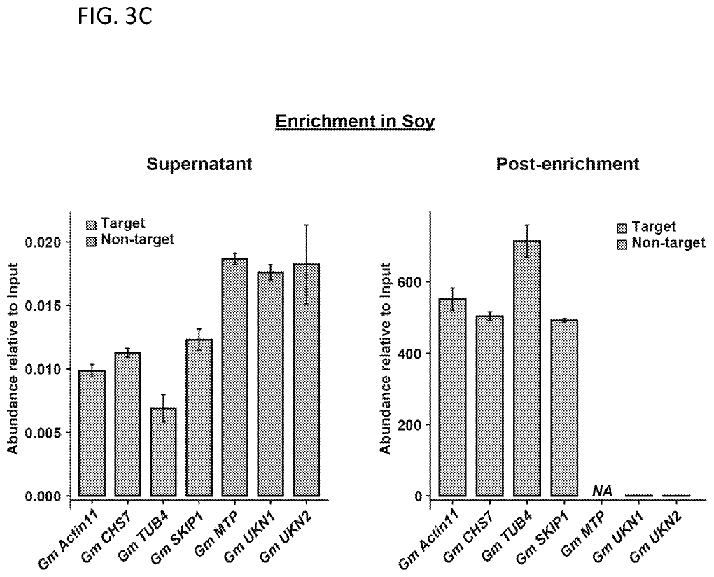

Following the generation of the cDNA libraries, All-In-One RNA-seq comprises sequencing cDNAs of one or more RNA molecules transcribed from the at least one pre-selected locus. In some aspects, sequencing is long-read sequencing. In some aspects, target molecule enrichment is accomplished by target capturing the specific sequences of interest out of the plurality of cDNA libraries using oligonucleotide probes to which the cDNA is hybridized, captured and thereby enriched (Step 5 in FIG. 1). Upon target capturing, the genes that are on the target list are enriched, whereas the genes that are not on the target list are reduced to undetectable levels (FIG. 3A-C). The oligonucleotide probes used in this enrichment step can target cDNAs of various RNAs and RNA variants described herein above. In some aspects, the oligonucleotide probes target endogenous RNAs such as transposable elements, protein-encoding genes, or non-coding RNAs. In other aspects, the oligonucleotide probes target exogenous RNAs such as pest, pathogen, or transgene RNAs.

Oligonucleotide probes suitable for target capture are known in the art. In some aspects, the oligonucleotide probes used for target capture comprise biotin modifications, thus the cDNA may be captured by biotinylated oligonucleotide probes, and subsequently isolated by magnetic streptavidin beads, washed, and eluted after hybridization.

Following the enrichment, the target captured cDNA is further prepared for long-read sequencing in the all-in-one RNA-sequencing assay disclosed above and herein. The preparation includes, but is not limited to, end-repairing the cDNA and ligating on adapter sequences (Step 6 in FIG. 1).

Lastly, All-In-One RNA-seq comprises sequencing the captured cDNA to obtain long reads representing full-length transcripts, thereby providing a sequence for each of the RNA molecules that is target captured from the original RNA sample (Step 6 of FIG. 1). Any third-generation sequencing is suitable for this assay. By way of non-limiting example only, the captured cDNA may be subject to Oxford Nanopore Technologies (ONT)-based sequencing to obtain long reads. The ONT-based sequencing includes, but is not limited to, using a R9.4.1 or a R10.1 flowcell on the MinION.

All-In-One RNA-seq can be used to analyze any organism that generates RNA. Suitable organisms include, but are not limited to a plant, animal, fungus, protist, bacterium, archaeon, and virus. By way of non-limiting example only, a plant may be Arabidopsis, corn, soybean, or rice. For example, any Arabidopsis, maize and/or soybean genotypes can be assayed to characterize transcriptionally active transposable elements (“TEs”, otherwise referred to in the literature as transposons, or jumping genes, and which produce polyadenylated and non-polyadenylated mRNAs), identify mutant alleles with known transgene insertions, and identify other transgenes of known transcriptional active or inactive states. Overall, the assay can allow characterization of gene- and TE-like transcriptional patterns and features in any organism including but not limited to the plant species listed herein.

### II. Libraries

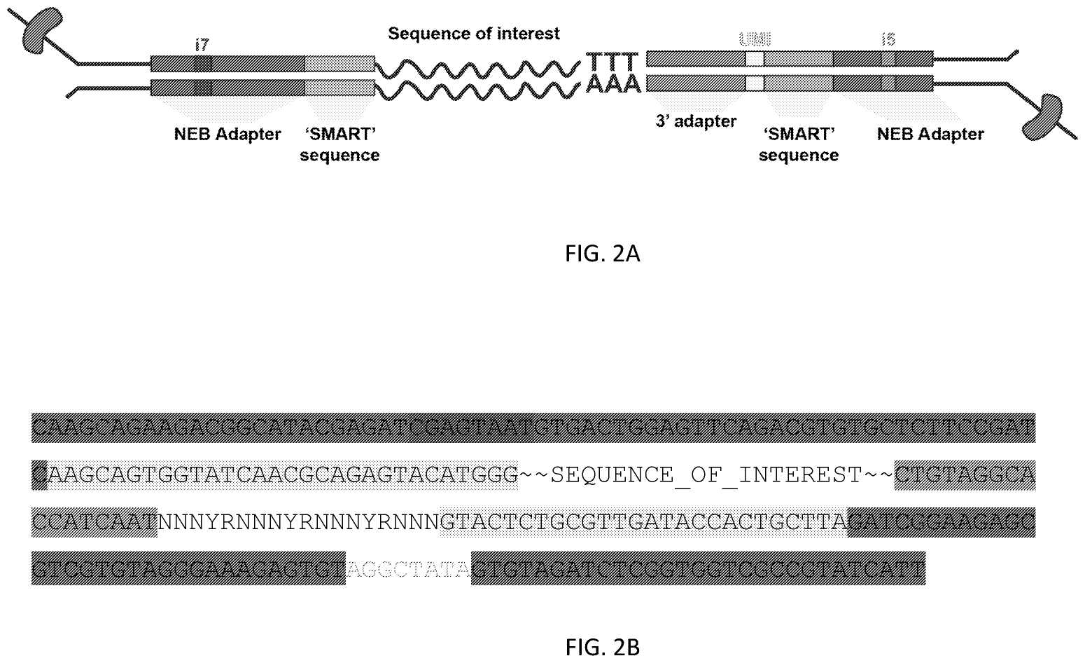

Another aspect of the instant disclosure encompasses a library of cDNAs each comprising a unique tag. The cDNAs can also comprise a multiplex index sequence identifying a library/sample. In some aspects, each cDNA in the library of cDNAs comprises a UMI unique tag and a multiplex index sequence identifying the library/sample (See FIG. 2 for sequences of full adapters added to the ends of each RNA molecule during cDNA sequence library production). The library can be pooled and amplified to facilitate subsequent manipulation. As explained herein above in Section I, the cDNA library can be amplified using primers that do not comprise unique tags, thus permitting amplification of all cDNAs at the same time.

Another aspect of the instant disclosure encompasses a pooled plurality of cDNA libraries, wherein each library is generated from RNA samples wherein each library comprises the full complement of cDNAs in a sample, wherein each sample comprises a UMI, and wherein each library comprises a unique index.

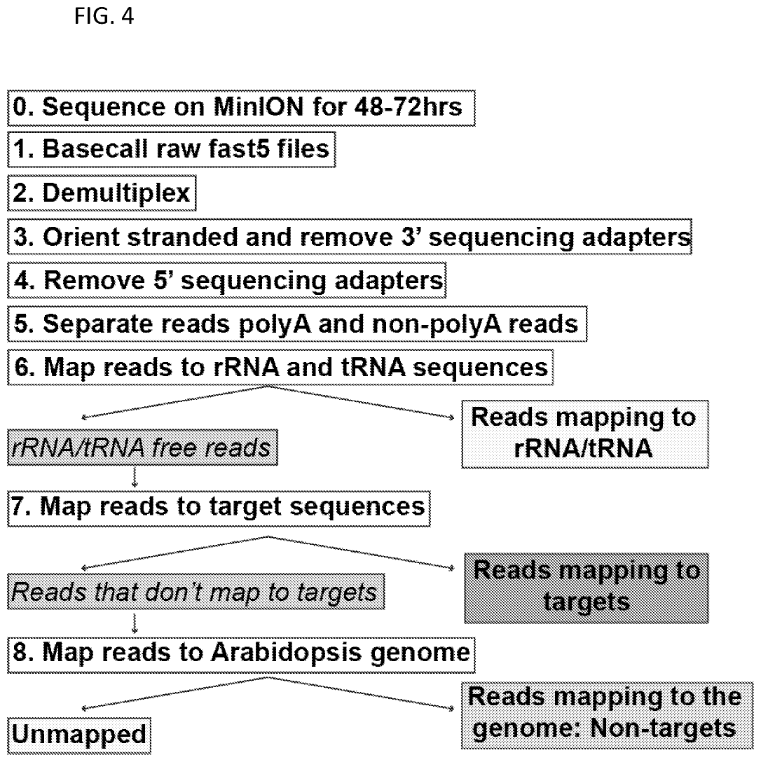

Data obtained from sequences (reads) can then be processed to characterize and quantitate RNAs transcribed from pre-selected loci. Processing reads from preselected loci obtained from pooled libraries can comprise one or more of demultiplexing the pool into individual libraries; informatically removing sequencing adapters that may have been used to obtain long read sequences (FIG. 2), separating the reads into polyadenylated and non-polyadenylated RNA, and orienting the long reads to the correct stand of RNA that is present in the organism (FIG. 4). The processing step optionally further comprises any one or all of mapping the reads to the rRNA and tRNA sequences to remove all contaminant sequences; mapping the reads that do not map to the rRNA/tRNAs to the target capture sequences; mapping the reads that do not map to the target capture sequences to the entire genome of the organism; and/or calculating the frequency of 5′ transcript start sites (TSSs), 3′ transcript termination sites (TTSs), splicing pattern, length of poly (A) tail and 3′ polyadenylation sites for the locus. In some aspects, the processing step may further comprise determining the features of RNA products such as but not limited to the quality and stability of the RNA products. Quality and stability of the RNA products may be determined by metrics including, but not limited to, determining the amount and/or proportion of RNA that is full-length and polyadenylated, the size of the region where polyadenylation occurs, the amount of sense vs. antisense RNA, the splicing pattern, the fit to periodicity of the known pattern of RNA degradation occurring at the 3′ ends of the exons, and/or the length of the poly (A) tail.

### III. Methods

Another aspect of the present disclosure encompasses a method of detecting or predicting stability of gene expression at a pre-selected locus or loci of an organism's genome. This method comprises sequencing total RNAs or a set of RNAs from the pre-selected locus or loci using the all-in-one RNA-sequencing assay as described in Section I herein above and processing the long reads to determine gene expression stability.

In another aspect, the gene is a transgene and determination of the transgene expression stability leads to prediction of future stability of the expression from the transgene in descendant plants when made homozygous, crossed into different lines, or subjected to post-transcriptional silencing, transcriptional epigenetic silencing or environmental stress. Organisms can include Arabidopsis, maize and/or soybean transgenic lines, and the assay is used to characterize transgene transcriptional patterns and features in these species.

Another aspect of the present disclosure encompasses a method of fast-tracking a transgenic event by selecting a stable transgenic event that has the most gene-like transgene expression patterns by using the all-in-one RNA-sequencing assay disclosed above and herein. By way of non-limiting example only, the gene-like transgene expression patterns include, but are not limited to, only minor production of antisense RNA, accurate transcriptional start sites, patterns of intron splicing, poly (A) tail length and/or clustering of polyadenylation sites. For instance, any transgenic plant such as, but not limited to Arabidopsis, maize and/or soybean transgenic lines can be assayed to fast-track stable transgenic events.

Still another aspect of the present disclosure encompasses a method of identifying off-type RNAs that trigger RNA decay, RNA degradation, transcriptional or post-transcriptional silencing. This method comprises sequencing total RNAs or a set of RNAs using the all-in-one RNA-sequencing assay as described herein; and processing the long reads to identify off-type RNAs.

A still further aspect of the present disclosure encompasses a method of diagnosing or characterizing a disease in an organism. This method comprises sequencing total RNAs or a set of RNAs from the organism using the all-in-one RNA-sequencing assay as described herein; and comparing the long reads to one or more reference RNA to identify irregularities in the total RNA or the set of RNAs indicative of the presence of a disease in the organism. The organism can be but is not limited to a human. For example, human RNA can be obtained to analyze regions of the genome that are associated with a disease of interest using All-In-One RNA-seq. Irregularities in the total RNA that may be identified include RNA degradation, RNA instability, incorrect RNA splicing, incorrect RNA processing, alternative transcriptional start or termination sites, shortening of poly (A) tail length and/or RNA decay. For instance, the assay can deeply study the RNA products of specific regions of the human genome that are highly studied for their role in disease, such as BRCA1.

### IV. Kits

A further aspect of the present disclosure provides kits for generating cDNA sequencing libraries. The kits can comprise adapters comprising unique tags for generating full-length cDNA transcripts comprising a unique tag identifying each RNA, adapters comprising unique indices, primers for generating cDNAs, primers for amplifying libraries, sequencing adapters and primers, or any combination thereof. Methods of generating libraries can be as described in Section I herein above.

The kits can further comprise reagents for generating the libraries such as reagents for ligating adapters, reagents for amplification of libraries, and sequencing reagents. The kits provided herein generally include instructions for carrying out the methods detailed below. Instructions included in the kits may be affixed to packaging material or may be included as a package insert. While the instructions are typically written or printed materials, they are not limited to such. Any medium capable of storing such instructions and communicating them to an end user is contemplated by this disclosure. Such media include, but are not limited to, electronic storage media (e.g., magnetic discs, tapes, cartridges, chips), optical media (e.g., CD ROM), and the like. As used herein, the term “instructions” may include the address of an internet site that provides the instructions.

### Definitions

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them unless specified otherwise.

When introducing elements of the present disclosure or the preferred aspects(s) thereof, the articles “a”, “an”, “the” and “said” are intended to mean that there are one or more of the elements. The terms “comprising”, “including” and “having” are intended to be inclusive and mean that there may be additional elements other than the listed elements.

As used herein, the term “library” refers to a collection of entities, such as, for example, cDNAs generated from an RNA sample. A library can comprise at least two, at least three, at least four, at least five, at least ten, at least 25, at least 50, at least 102, at least 103, at least 104, at least 105, at least 106, at least 107, at least 108, at least 109, or more different entities. In some aspects, a library refers to a collection of nucleic acids that are propagatable, e.g., through a process of clonal amplification. Library entities can be stored, maintained, or contained separately or as a mixture.

As used herein, the term “endogenous” as in the phrase “endogenous genes” refers to genes that are native to an organism of interest and are originating or developing within the organism. Non-limiting examples of endogenous RNAs include, but are not limited to, transposable elements, protein-encoding genes, and non-coding RNAs.

As used herein, the term “exogenous” as in the phrase “exogenously introduced genes” refers to gees that are not native to the organism of interest. Non-limiting examples of exogenous genes include, but are not limited to, pest, pathogen and transgene RNAs.

As used herein, the terms “polypeptide” and “protein” are used interchangeably to refer to a polymer of amino acid residues.

As used herein, the terms “upstream” and “downstream” refer to locations in a nucleic acid sequence relative to a fixed position. Upstream refers to the region that is 5′ (i.e., near the 5′ end of the strand) to the position, and downstream refers to the region that is 3′ (i.e., near the 3′ end of the strand) to the position.

As used herein, the term “gene” refers to a segment of DNA that contains all the information for the regulated biosynthesis of an RNA product, including promoters, exons, introns, and other untranslated regions that control expression.

As used herein, the terms “all-in-one RNA-seq”, “all-in-one RNA-sequencing”, and “All-In-One RNA-seq” refer to the assay disclosed above and herein, wherein full-length RNA transcripts are obtained using qualitative long-read sequencing that incorporates molecular indices to quantitatively count reads. Such assay provides a qualitative characterization and quantitative measurement of all RNAs for a select locus or select loci.

As used herein, the term “pooling” refers to combining multiple libraries before sequencing, each with a unique molecular barcode (or unique combination of multiple barcodes) to keep sequencing cost-effective. The sequencer then reads each library molecule's biological base sequence as well as the barcode sequence; these barcodes are matched back to the sequences expected from the libraries, and thus each molecule can be attributed to its library of origin even though the libraries are mixed.

As used herein, the term “demultiplexing” refers to a step of processing sequence data obtained from multiple libraries that are pooled, wherein reads from individual libraries are analyzed due to the use of a unique molecular barcode (or unique combination of multiple barcodes) specific for each library.

As used herein, the term “target capture” refers to a process before long-read sequencing that enriches the genes that are on the target list, whereas the genes that are not on the target list are reduced to undetectable levels. In this process, specific sequences of interest out of the plurality of cDNA libraries are targeted using oligonucleotide probes to which the cDNA is hybridized, captured and thereby enriched.

As used herein, the term “read” is an inferred sequence of base pairs corresponding to all or part of a single DNA fragment. The terms “long reads” and “long-read sequences” refer to sequences of DNA between 1,000 and 100,000 base pairs in length Long reads allow for more sequence overlap, thus useful for de novo assembly and resolving repetitive areas of the genome with greater confidence.

As used herein, the term “long-read sequencing” refers to a DNA sequencing technique that can determine the nucleotide sequence of long sequences of DNA between 1,000 and 100,000 base pairs at a time.

As used herein, the terms “off-type RNAs” or “aberrant RNAs” refer to RNAs that contain premature termination codons (aberrantly spliced RNAs) and have characteristics of nonsense-mediated decay substrate. Off-type or aberrant RNAs may cause gene silencing, thus detection of such RNAs may predict the future stability of gene expression from pre-selected locus.

### EXAMPLES

As various changes could be made in the above-described assay and methods without departing from the scope of the invention, it is intended that all matter contained in the above description and in the examples given below, shall be interpreted as illustrative and not in a limiting sense.

The following examples are included to demonstrate the disclosure. It should be appreciated by those of skill in the art that the techniques disclosed in the following examples represent techniques discovered by the inventors to function well in the practice of the disclosure. Those of skill in the art should, however, in light of the present disclosure, appreciate that many changes could be made in the disclosure and still obtain a like or similar result without departing from the spirit and scope of the disclosure, therefore all matter set forth is to be interpreted as illustrative and not in a limiting sense.

### Example 1: Selection of Target Loci

The first step of an all-in-one RNA-seq is to select the loci (genes or non-protein coding regions) that the user wants to investigate. Fifty (50) loci were selected from the Arabidopsis thaliana genome, including protein coding genes, transposable element (TE) loci, phasiRNA loci and promoters/coding regions/terminators that have been used in transgenic Arabidopsis lines. The company Daicel Arbor BioSciences used their service “MyBaits” to synthesize ˜10,000 single-stranded DNA oligonucleotides, 80 nucleotides in length, complementary to the 50 loci selected in this study. These oligonucleotides have biotin incorporated, so target capture of a DNA (or cDNA in this study) library can be performed before sequencing. It was not important which strand the oligos were generated from, since the RNA would be converted into double-stranded cDNA before target capture was performed.

In other experiments a different target capture set for Arabidopsis that included a different transgene sequence that was integrated into the Arabidopsis genome, called RUBY was generated. In other experiments a different target capture set with regions from both the maize (Zea mays) and soybean (Glycine max) genomes were generated to expand the all-in-one sequencing technique into major crop species of interest. The regions of the genome included endogenous protein-coding genes from both species, transposable element and expressed non-protein-coding regions from each genome, as well as the sequences of transgenes that have been placed into the maize and soybean genomes.

### Example 2: All-In-One RNA-Seg Library Production

All-in-one RNA-seq libraries were produced from Arabidopsis inflorescences (flower buds), Arabidopsis leaves, maize leaves, soybean leaves and mature flower tissue. Other samples processed using this ‘All-in-one’ RNA-seq methodology are a reference strain of maize, a reference strain of soybean, five lines of soybean that had a transgene integrated into their genomes, and more Arabidopsis plants with the RUBY transgene. The production of all-in-one RNA-seq libraries from Arabidopsis inflorescences are described here, but the other libraries were produced using similar methods.

For Arabidopsis, two biological replicates of wild-type stain Columbia were used as a reference. In addition, mutants lacking RNA Polymerase IV (pol IV′), RNA polymerase V (pol), a double-mutant combination of pol IV/pol V′, Decrease in DNA Methylation 1 (ddm1), Argonaute 6 (ago6), or RNA-dependent RNA Polymerase 6 (rdr6) as well as two transgenic lines including the ago6 mutant with a complementing transgene of FLAG-AGO6, and the rdr6 mutant with a complementing transgene of RDR6-GFP.

TRIzol reagent (Thermo Fisher Scientific) was used to isolate RNA (see, step 1 in FIG. 1), and a commercially available modified RNA oligonucleotide was ligated onto the 3′ of each RNA molecule (New England Biolabs Universal miRNA Cloning Linker) (see, step 2 in FIG. 1), as in (Jia et al., Nature Plants, 6 (7): 780-788, 2020). This ligation occurred on polyadenylated, non-polyadenylated and partially degraded RNAs. The Takara SMARTer cDNA Synthesis Kit was used to convert the RNA into cDNA with one major modification. Specifically, instead of the provided oligo-dT primer, a custom DNA oligonucleotide that is complementary to the Universal miRNA Cloning Linker and contains a Unique Molecular Index (UMI) was used (see, step 3 in FIG. 1). An aspect of the primer for cDNA comprises the following sequence: AAGCAGTGGTATCAACGCAGAGTACNNNYRNNNYRNNNYRNNNATTGATGGTGCCT ACAG (SEQ ID NO: 2). This modification allowed conversion of all RNAs that had the 3′ adapter ligated into cDNA (both polyadenylated and non-polyadenylated RNA) and also addition of an 18-nucleotide sequence molecular tag (UMI). By doing this, each individual cDNA molecule had a unique tag that allowed for the determination of which sequencing reads were PCR duplicates of each other (they had the same UMI), resulting in quantification of cDNA molecules post-amplification and hence RNA molecules in the cell. UMIs that are best for ONT sequencing were taken from Karst et al. (Nature Methods, 18 (2): 165-169, 2021).

The double stranded cDNA (ds-cDNA) generated above was used as input for a traditional Illumina DNA sequencing library preparation using the NEBNext DNA library kit for Illumina (see, step 4 in FIG. 1). This kit first end-repaired the cDNA molecules before it ligated DNA adapter sequences to the ends of each cDNA molecule to allow for a subsequent PCR to add standard 8-nucleotide Illumina multiplex indices that were unique for each library/sample. These indices are essential, as it permits pooling of several libraries together before the target capture enrichment, making this protocol extremely scalable. At this step, ds-cDNA libraries possessed two essential features: 1) a UMI to help distinguish PCR duplicates and enable quantification of long-read RNA-seq and 2) a multiplex index sequence to permit pooling and subsequent demultiplexing (FIG. 2A). FIG. 2B shows the sequence of an aspect of a long read sequence using the assay of the instant disclosure including the Illumina adapters from the NEBNext Ultra II DNA library preparation kit, the ‘SMART’ sequence from the Takara SMARTer cDNA synthesis kit, the NEB Universal miRNA Cloning Linker, the UMI is in Yellow, and i7 and i5 indices (SEQ ID NO: 1 represents the sequence left of sequence of interest; SEQ ID NO: 2 represents the sequence right of sequence of interest).

Once PCR amplified, the indexed libraries were pooled and subjected to target capture with the Daicel Arbor BioSciences set of biotin-labeled oligos (see, step 5 in FIG. 1). In particular, the cDNA was hybridized to the oligos, captured using magnetic streptavidin beads, washed and eluted. Quality-control experiments demonstrated a high level of target enrichment using this approach (FIGS. 3A-3C). After target capture, the library was amplified again using primers that do not contain indexes or UMIs, permitting amplification of all pool libraries at the same time. The library was controlled for quantity and quality, and subjected to the ONT DNA Ligation Sequencing Kit (Oxford Nanopore Technologies), and sequenced on the ONT platform using a R9.4.1 flowcell on the MinION (see, step 6 in FIG. 1).

### Example 3: Read Processing

Sequencing generates 25-35 million reads per ONT flowcell that average 567-1058 nucleotides in length (FIG. 4, step 0). In addition, samples can be run on multiple ONT flowcells if more reads are needed. After sequencing, the reads produced from the MinION must first be converted from fast5 to fastq file format (FIG. 4, step 1). This step is extremely computationally intensive and is essential to permit downstream analyses. Because there is not an established pipeline to process this data type, significant computational analysis and testing was necessary to demultiplex the pool into individual libraries and orient the reads to the correct stand of RNA that was present in the cell (FIG. 4, step 2). A significant bioinformatics challenge was overcome to demultiplex the samples without removing up to 40% of the data that originally could not be resolved.

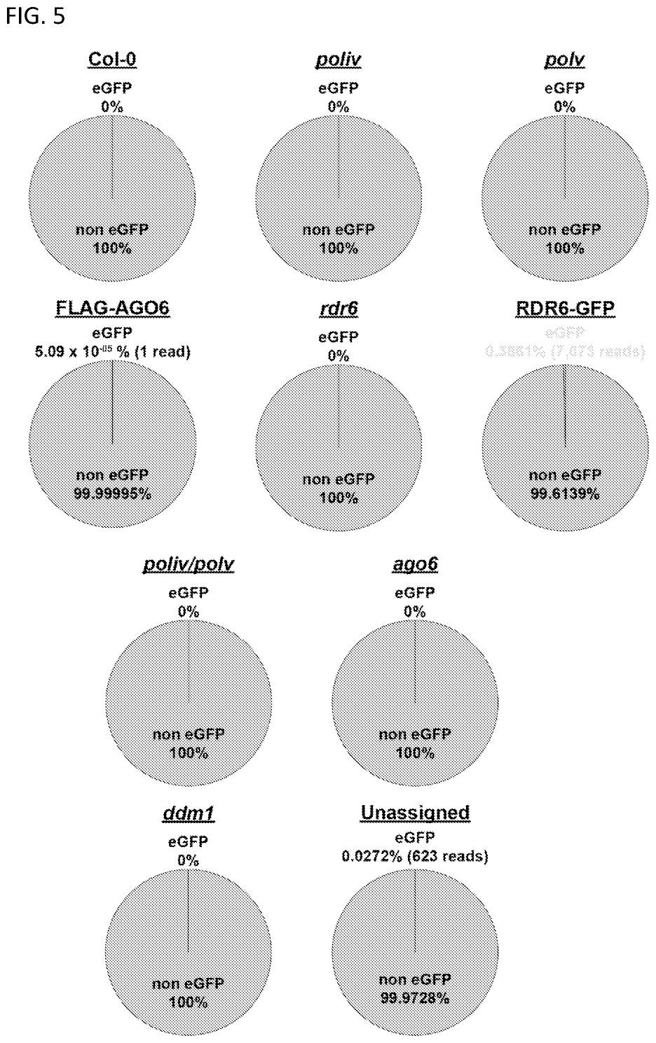

To determine the accuracy of the demultiplexing pipeline, a control experiment was performed to ensure the mapping and assigning of pooled reads are demultiplexed into the correct sample wherein only one genotype should contain reads mapping to eGFP. This result shows that the demultiplexing of pooled samples occurs very accurately (FIG. 5).

Once the reads were confidently demultiplexed, the sequencing adapters at the very ends of the library (see, FIG. 2) were informatically removed. This had two main functions: 1) allowing easier mapping to the genome, and 2) orienting the reads to determine which strand of DNA the RNA transcript was generated from. Since these libraries contained both polyadenylated and non-polyadenylated RNA, the next step was to separate the reads into these two subgroups for downstream analyses (FIG. 4, step 5).

For post-processing, the reads were first mapped to the rRNA and tRNA sequences to remove all contaminant sequences that were not of interest in this experiment (FIG. 4, step 6). Reads that did not map to the rRNA/tRNAs were then aligned to the target captured set of loci (FIG. 4, step 7). Reads that didn't map to the targeted loci were then mapped to the entire Arabidopsis genome (FIG. 4, step 8). Once all reads were mapped and classified, the location and precision of 5′ transcript start sites (TSSs), 3′ transcript termination sites (TTSs) and 3′ polyadenylated sites for each locus could be examined and calculated. Metrics such as percent of transcripts that are full-length and polyadenylated, so called ‘translatable’ RNAs, percent of transcripts that are mapped to the sense vs antisense strand, and the percent of reads that map to the introns vs exons of protein-coding genes and transgenes, to name a few, can be calculated. Between 10,000 and 800,000 transcripts per gene target were obtained after filtering and processing.

### Example 4: Identification of Gene-Like Features

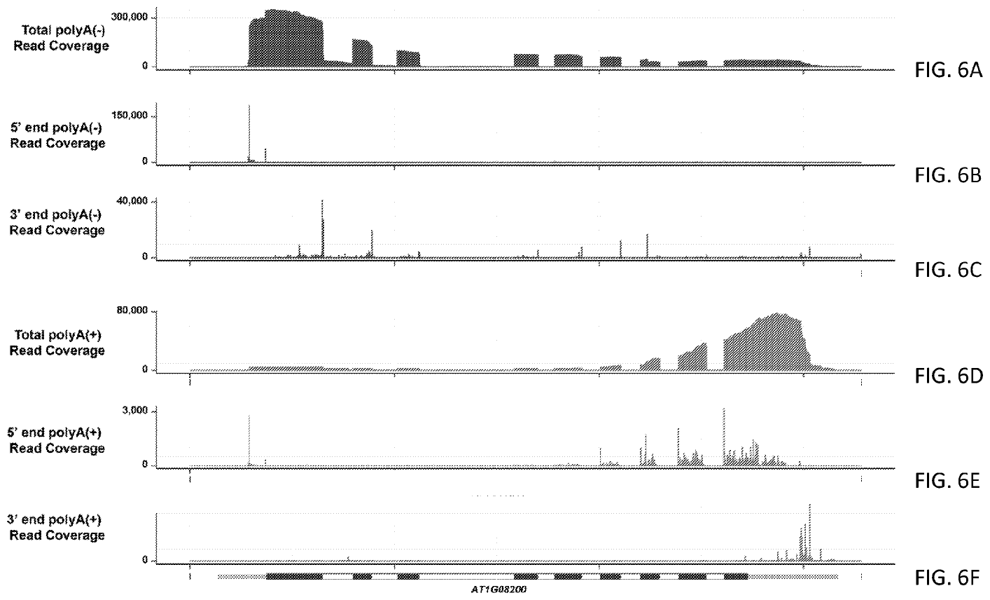

Observation of the genes on the target capture list demonstrated several key features signifying that this region of the genome produced a protein-coding mRNA without inhibition from transcriptional or post-transcriptional forms of gene silencing. First, the non-polyadenylated RNAs revealed the expected splicing pattern of the gene, with the bulk of the reads accumulating in the 5′ region and clear read deficits in the intronic regions that have been spliced out (FIG. 6A). This data also reveals a buildup of reads in the first intron, a pattern that indicates partial splicing of intron 1, which is known to be the slowest to be spliced out (Herzel et al., Genome Research, 28 (7): 1008-1019, 2018; Drexler et al., Molecular Cell, 77 (5): 985-998, 2020). It suggests that RNAs that were actively being transcribed were being identified. Second, there was strong enrichment of the 5′ end of reads at one site, suggesting the true transcription start site (TSS) (FIG. 6B). This site is often annotated in the available genome data incorrectly, and the all-in-one RNA-seq assay provides this information with high accuracy. While the annotation depicted did not directly align with the experimental TSS, several analyses of the 5′ end of transcripts (CAGE data published on JBrowse) matched up exactly with the experimental data obtained herein. Third, there is the accumulation of non-polyadenylated reads at the 3′ end of the exon (5′ splice site), i.e., the enrichment of 3′ end of reads at the 5′ splice site (FIG. 6C). This pattern has been observed before in both animal and plant systems (Jia et al., Nature Plants, 6 (7): 780-788, 2020; Herzel et al., Genome Research, 28 (7): 1008-1019, 2018; Drexler et al., Molecular Cell, 77 (5): 985-998, 2020) and provides a predictable periodicity to calculate the expected vs. observed pattern of RNA decay. Interestingly, these reads appeared in a predictable pattern at the 3′ end of each exon (FIG. 6C).

With the polyadenylated transcripts, there was again a strong signature of splicing, but now a build-up of poly (A)+reads in the 3′ end of the gene (FIG. 6D). These reads started at the TSS, while there was also a pattern of RNA decay at the 5′ ends of exons (FIG. 6E). Strong peak at the 5′ end of reads at the TSS suggested that some full-length, polyadenylated RNAs were being observed, while many different 5′ ends throughout the 3′ end of the transcript indicated normal decay intermediates from 5′->3′. Finally, there was a region of polyadenylation on the 3′ UTR (FIG. 6F). Various 3′ end of reads within the 3′ UTR suggested that there was more than one site of polyadenylation, but they were all concentrated in close proximity.

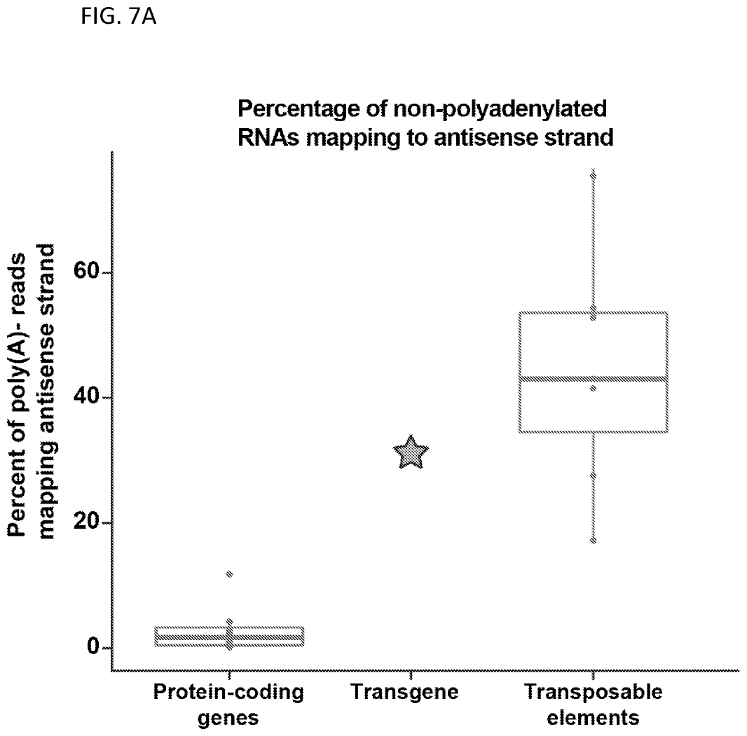

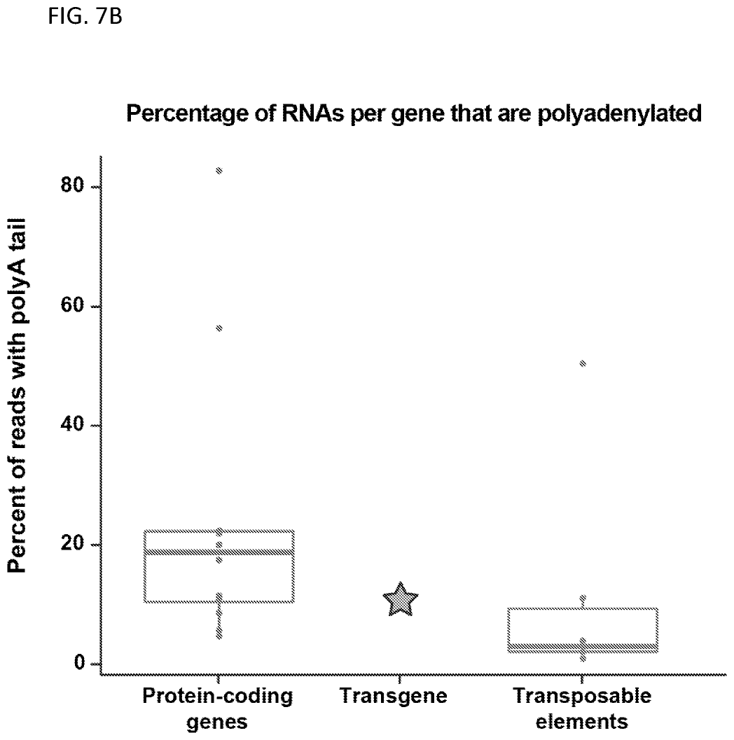

Looking at the gene-set as a whole group, it was observed that the amount of antisense transcripts compared to sense was very low (˜5%) for protein-coding genes (FIG. 7A), and the amount of polyadenylated to non-polyadenylated transcript was surprisingly typically under 20% for genes (FIG. 7B).

### Example 5: Identification of Transposable Element-Like Features

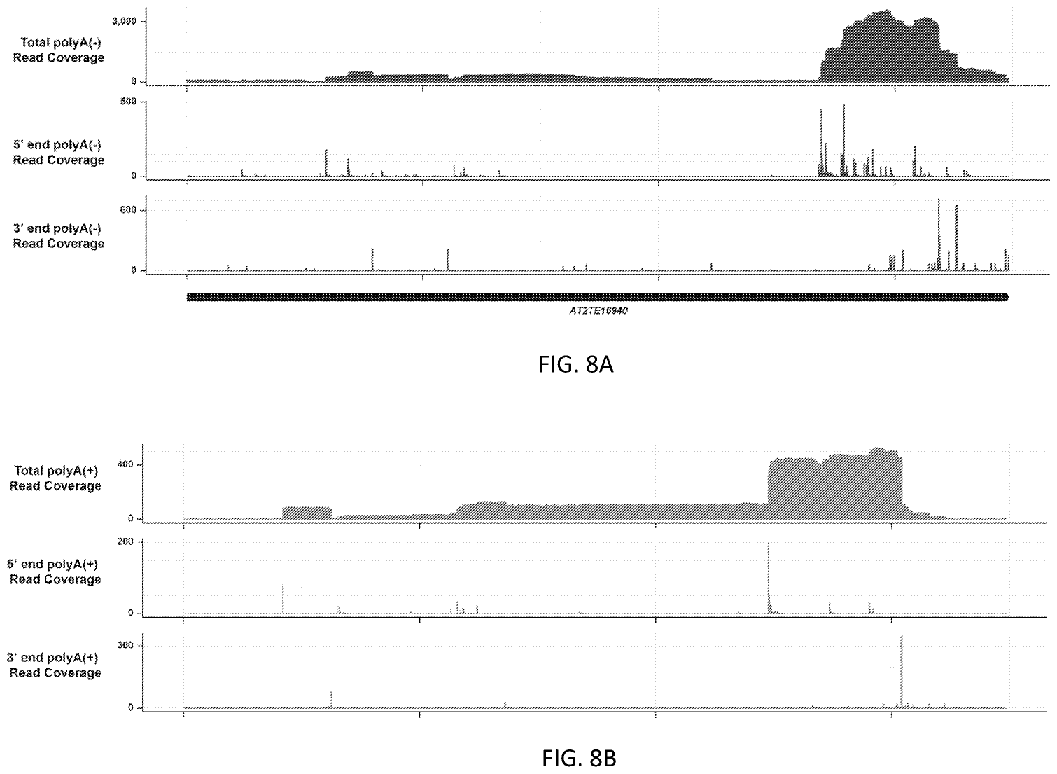

Unlike the genes, the transposable elements (TEs) on the target capture array as a group produced much higher levels of antisense RNA and much lower levels of polyadenylated mRNA, corresponding to the transcriptional-level and post-transcriptional-level silencing of mRNA production from these loci (FIGS. 7A-7B). Additionally, TEs lacked many features that were observed in the protein-coding RNAs, including loss of buildup of reads in the 3′ of the element, no strong 5′ TSS peak, and a weak polyadenylation site. As shown in FIG. 8A, a close examination of non-polyadenylated read accumulation revealed TE-like features such as no strong 5′ peak indicating the TSS for non-poly (A) reads, and a slight buildup of reads at the 3′ end. As shown in FIG. 8B, a close examination of polyadenylated read accumulation revealed TE-like features such as a only a weak peak at the true TSS for poly (A) reads, but a strong “gene like” 3′ end peak indicating a poly (A) site.

### Example 6: Identification of Transgene Features

The transgene produced RNAs that displayed intermediate levels between what was observed for the protein-coding genes and for the TEs as far as antisense production and percentage of polyadenylated reads generated (FIGS. 7A-7B). This is true for Arabidopsis transgenes (FLAG-AGO6, RDR6-GFP and RUBY), as well as investigated soybean transgenes. Two key metrics that were found to analyze are how much RNA is full-length and polyadenylated for the transgene, and how much antisense RNA is the transgene making.

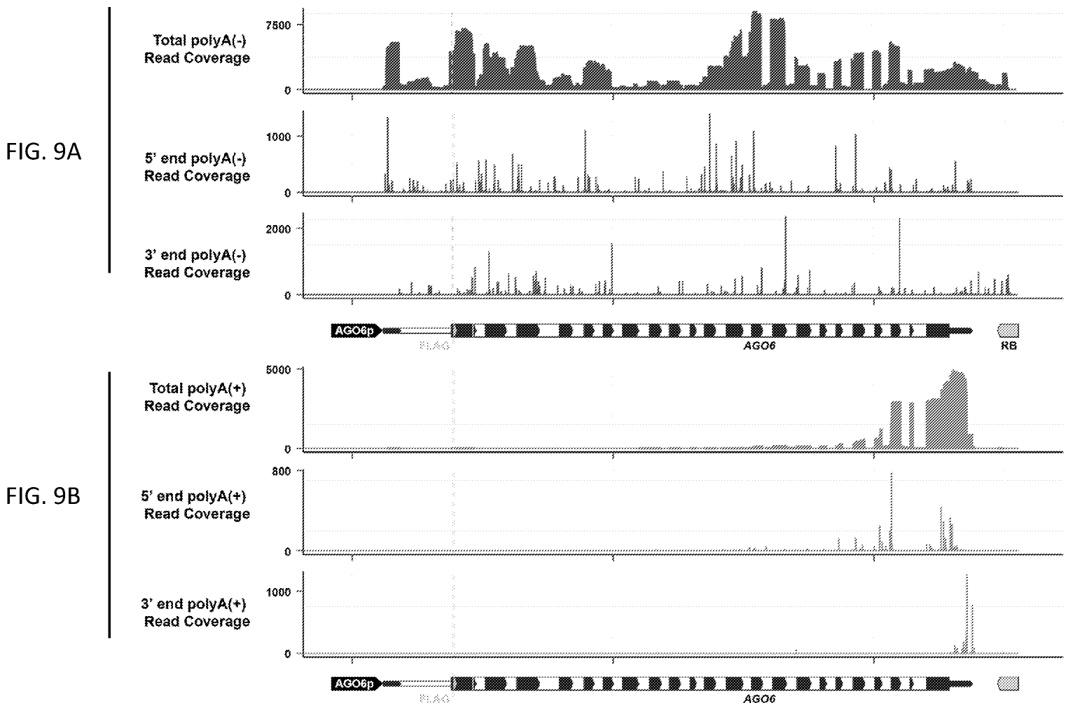

A close examination of non-polyadenylated read accumulation revealed that transgene RNAs possessed some gene-like features such as a peak of 5′ ends of reads at the TSS and a buildup of 3′ ends of reads at the 5′ splice sites (FIG. 9A). However, the transgene generated RNAs that also have some TE-like (non-gene) features such as reads mapped to many introns (not just the first intron), and wide-spread 5′ and 3′ ends of reads throughout the entire gene (FIG. 9A). A close examination of polyadenylated read accumulation revealed that transgene RNAs possessed some gene-like features such as various 3′ end of reads within the 3′ UTR and a buildup of poly (A)+reads in the 3′ end of the gene (i.e., polyadenylation occurring in the proper location) (FIG. 9B). However, the transgene generated RNAs that also have some TE-like (non-gene) features such as no strong peak in the 5′ end of reads at the TSS (FIG. 9B). Together, this demonstrates that although a transgene may be functional, generate sufficient protein and complement the corresponding mutation (McCue et al., The EMBO Journal, 34 (1): 20-35, 2015), it did not fully trick the cell's transcription and RNA processing machinery into believing that it was a gene. Even though the transgene is functional, it is generating RNA transcripts subject to processing and degradation that will reduce the transgene's future stability of expression. Therefore, the All-in-One RNA-seq expression profile is predictive of future transgene expression stability.

### Example 8

Removal of ribosomal RNA from the total RNA before library production was attempted. However, Ribosomal RNA depletion did not reduce the number of Ribosomal RNA reads in the final library, informing us that we did not need to do this step again. This is important because this step is costly and time-consuming. Instead, the target capture step removed nearly all ribosomal-derived cDNA molecules from the sequencing reaction.

When performing experiments in maize and soy, the full sequence of the transgenes that had been added to those genomes was added to the target capture list, in order to enrich for and examine RNAs from these transgenes. However, several of these transgenes contained tRNA sequences, which are used as a platform to process multiple CRISPR guide RNAs from one transcript in the transgene. When target capture was performed using these sequences, a large amount of endogenous tRNA molecules that had been accidentally enriched and sequenced was identified, since they match the target capture sequence. Although this outcome was not sought, it does prove that tRNA molecules can be processed into All-in-One sequencing libraries, enriched and sequenced in this manner, which is important for studying tRNA transcripts.

### Example 9

All-In-One RNA-seq may have various applications. For example, any biologist that investigates the RNA pattern from one locus or a set of loci will be able to use this assay to obtain a much higher qualitative resolution by looking at the RNAs generated and decayed from the locus/loci of interest.

Next, plant biologists may perform this assay on a few or many individual transgenic lines to determine if the transgene they have engineered has a gene-like transcriptional profile. This could be important not only for the plant under direct study, but also to predict the future stability of the expression from this transgene in descendant plants with this transgene when made homozygous, crossed into different lines, or subjected to environmental stress.

Additionally, All-In-One RNA-seq may be used to identify uncharacterized “aberrant” RNAs that trigger transcriptional or post-transcriptional silencing. Since it is known that silencing is triggered on the RNA level (Fultz and Slotkin, The Plant Cell, 29 (2): 360-376, 2017), off-type RNAs have been blamed for decades as the reason why a gene or transgene is silenced, but the state of art does not understand what an aberrant RNA is (e.g., the features it has that trigger silencing).

Further, disease researchers may perform this assay to determine if a problem exists in a patient or sample, such as incorrect RNA splicing, processing or RNA decay.

Moreover, All-In-One RNA-seq may be used to diagnose or characterize diseases where there is no change in DNA (or difficult to assay), but the problem can be observed more easily on the RNA level.

Still moreover, researchers may be able to better quantify the level of RNA accumulation from their loci of interest using All-In-One RNA-seq since they will be able to determine how many transcripts are full-length as well as to completely collapse PCR duplicates using the UMIs.

Discussion: when performing RNA sequencing, either in an academic lab or industry, to investigate the RNA levels of a transgene, a gene or a set of genes of interest, one has to choose which types of RNA they want to detect due to the limitations of available methods. For example, most “RNA-seq” currently available focus on sequencing only mRNA. However, it has been demonstrated that other (i.e., non-polyadenylated) RNAs are the triggers for silencing. These other RNAs are often not captured in traditional RNA-seq experiments because they are 1) low abundance, 2) not uniform, and 3) not polyadenylated mRNAs. A technique to thoroughly detect all RNAs coming from a transgene, a gene or a set of genes of interest was needed.

The present disclosure provides an all-in-one RNA-sequencing assay, which is better than traditional RNA-seq in that it captures all RNAs from the locus/loci of interest, including mRNA, non-polyadenylated RNA, and partially degraded RNA. It also provides a coverage of 10,000-800,000 full-length transcripts per gene, which is much higher than traditional RNA-seq. Further, it produces long-read sequences, so each read has information on transcriptional start-sites, termination sites, cleavage points, splicing, polyadenylation levels, and poly (A) tail length, all on a single read. Most long-read sequencing currently available in the art is only qualitative (not quantitative). In contrast, All-In-One RNA-seq is both qualitative and quantitative.

This assay has been performed on Arabidopsis, maize and soybean samples, which produced a very detailed view of the transcripts of selected 50 regions/loci of the genome. Genes have a very specific pattern, e.g., accurate transcriptional start sites, patterns of intron splicing, clustering of polyadenylation sites, whereas non-gene regions such as transposable elements do not have these gene-like signatures. Transgenes are a mixture of gene-like and non-gene like signatures, which account for their known instability.

All-In-One RNA-seq may be used as a tool to detect transgene expression stability, and further predict future late-stage failure by transgene silencing. It has been argued that if a transgene has poor gene-like transcriptional signatures, it will be a “fingerprint” of both current and future instability, that is, this event will enter a ‘no-go’ situation as it will silence in the future. All-In-One RNA-seq could be used to determine which transgenic events have the most “gene-like” transgene expression patterns and therefore, to fast-track those events.

All patents and publications mentioned in the specification are indicative of the levels of those skilled in the art to which the present disclosure pertains. All patents and publications are herein incorporated by reference to the same extent as if each individual publication was specifically and individually indicated to be incorporated by reference.

The publications discussed throughout are provided solely for their disclosure before the filing date of the present application. Nothing herein is to be construed as an admission that the invention is not entitled to antedate such disclosure by virtue of prior invention.

### SEQUENCES

| SEQ ID | |
|---|---|
| NO: | SEQUENCE |
| 1 | CAAGCAGAAGACGGCATACGAGATCGAGTAATGTGACTGGA |
| | GTTCAGACGTGTGCTCTTCCGATCAAGCAGTGGTATCAACG |
| | CAGAGTACATGGG |
| 2 | CTGTAGGCACCATCAATNNNYRNNNYRNNNYRNNNGTACTC |
| | TGCGTTGATACCACTGCTTAGATCGGAAGAGCGTCGTGTAG |
| | GGAAAGAGTGTAGGCTATAGTGTAGATCTCGGTGGTCGCCG |
| | TATCATT |
| 3 | AAGCAGTGGTATCAACGCAGAGTACNNNYRNNNYRNNNYRN |
| | NNATTGATGGTGCCTACAG |

What is claimed is:

1. An all-in-one RNA-sequencing assay, comprising:
a) ligating an RNA, DNA or synthetic adapter to the 3′ end of each RNA molecule among total RNAs or a set of RNAs molecules transcribed from at least one pre-selected locus of an organism's genome, to form ligated RNAs;
b) obtaining full-length cDNA transcripts using the ligated RNAs as input, wherein each cDNA transcript comprises a unique tag identifying each RNA;
c) generating a cDNA sequencing library using the cDNA transcripts, wherein all cDNAs in the sequencing library comprise a multiplex index identifying the library; and
d) sequencing cDNAs of one or more RNA molecules transcribed from the at least one pre-selected locus, thereby obtaining a sequence for each of the RNA molecules from the original RNA sample.

2. The assay of claim 1, wherein the full-length cDNA transcripts transcribed from the at least one pre-selected locus are obtained by reverse transcribing the ligated RNAs to obtain full-length cDNA transcripts, wherein each cDNA transcript comprises a unique tag.

3. The assay of claim 1 or claim 2, wherein sequencing specific cDNAs of transcripts transcribed from the at least one pre-selected locus RNA comprises target capturing specific sequences of interest out of pooled plurality cDNA libraries using oligonucleotide probes to which the cDNA is hybridized, captured, and thereby enriched.

4. The assay of claim 3, wherein the oligonucleotide probes target various endogenous RNAs or exogenous RNAs.

5. The assay of claim 4, wherein the endogenous RNAs comprise transposable elements, protein-encoding genes, and/or non-coding RNAs.

6. The assay of any of the preceding claims, wherein sequencing specific cDNAs of RNA molecules of interest comprises obtaining long reads representing full-length transcripts, thereby providing a long read sequence for each of the RNA molecules that is target captured from the original RNA sample.

7. The assay of any of the preceding claims, further comprising generating a plurality of cDNA libraries from a plurality of RNA samples, wherein each library comprises cDNAs comprising a multiplex index sequence identifying the library.

8. The assay of any of the preceding claims, wherein the total RNAs comprise polyadenylated RNA, non-polyadenylated RNA, partially degraded RNA, partially processed RNA, alternatively spliced variants of RNAs, or transcription start site variants of RNAs.

9. The assay of any of the preceding claims, wherein the at least one pre-selected locus comprises a transgene, a gene or a set of genes of interest, a pathogen, or pest sequence within a host organism.

10. The assay of claim 1, wherein the adapter is a DNA, RNA, synthetic adaptor, or a combination thereof, that is used to add a cDNA priming site to the 3′ ends of the RNAs.

11. The assay of claim 10, wherein the adapter is the Universal miRNA Cloning Linker.

12. The assay of claim 1, wherein a DNA oligonucleotide that is complementary to the 3′ adapter is used as a primer to reverse transcribe the RNAs to the cDNA.

13. The assay of claim 12, wherein the DNA oligonucleotide is complementary to the 3′ adapter and comprises the unique tag that is different for each cDNA molecule.

14. The assay of claim 12, wherein the unique tag is a Unique Molecular Index (UMI) tag.

15. The assay of claim 13, wherein the 3′ adapter is the Universal miRNA Cloning Linker.

16. The assay of claim 1, wherein the unique tag allows for distinguishing and collapsing PCR duplicates and enabling quantification of cDNA sequences.

17. The assay of claim 1, wherein the multiplex index sequence permits pooling and subsequent demultiplexing of the indexed cDNA libraries.

18. An all-in-one RNA-sequencing assay, comprising:
a) ligating an RNA, DNA or synthetic adapter to the 3′ end of each RNA molecule among total RNAs to form ligated RNAs;
b) reverse transcribing the ligated RNAs to obtain full-length cDNA transcripts, wherein each cDNA transcript comprises a unique tag;
c) generating a plurality of cDNA libraries, wherein each library contains a multiplex index sequence;
d) target capturing specific sequences of interest out of the plurality of cDNA libraries using oligonucleotide probes to which the cDNA is hybridized, captured, and thereby enriched; and
e) sequencing the captured cDNA to obtain long reads representing full-length transcripts, thereby providing a sequence for each of the RNA molecules that is target captured from the original RNA sample.

19. The assay of claim 18, wherein the exogenous RNAs comprise pest, pathogen or transgene RNAs.

20. The assay of claim 18, wherein the cDNA is captured by biotinylated oligonucleotide probes, and subsequently isolated by magnetic streptavidin beads, washed, and eluted after hybridization.

21. The assay of claim 20, further comprising amplifying the libraries using primers that do not contain the tags or the multiplex index sequences, permitting amplification of all pool libraries at the same time.

22. The assay of claim 18, wherein the step of preparing the captured cDNA comprises end-repairing the cDNA, ligating on adapter sequences, and amplifying the cDNA with primers that comprise the multiplex indexes for multiplexing.

23. The assay of claim 18, wherein the long-read sequencing comprises Oxford Nanopore-based sequencing of the cDNAs.

24. The assay of claim 18, wherein the organism is a plant, animal, fungus, protist, bacterium, archaeon, or virus.

25. The assay of claim 24, wherein the organism is a plant selected from the group consisting of Arabidopsis, corn, soybean, and rice.

26. A sequencing library of cDNAs each comprising a unique tag generated using an assay of claim 1.

27. The library of claim 26, wherein the cDNAs comprise a multiplex index sequence identifying a library/sample.

28. A pooled plurality of cDNA libraries generated using an assay of any one of claims 1-27, wherein each library is generated from an RNA sample, wherein each library comprises the full complement of cDNAs in a sample, wherein each sample comprises a unique tag, and wherein each library comprises a multiplex index.

29. A method of detecting or predicting stability of gene expression at a pre-selected locus of an organism's genome, the method comprising:
a) sequencing total RNAs or a set of RNAs from the pre-selected locus using the all-in-one RNA-sequencing assay of any proceeding claim; and
b) processing the long reads to determine gene expression stability.

30. The method of claim 29, wherein the processing step comprises:
a) demultiplexing the pool into individual libraries; and
b) orienting the long reads to the correct stand of RNA that is present in the organism.

31. The method of claim 30, further comprising:
a) mapping the reads to the rRNA and tRNA sequences to remove all unwanted or contaminant sequences;
b) mapping the reads that do not map to the rRNA/tRNAs to the target capture sequences;
c) mapping the reads that do not map to the target capture sequences to the entire genome of the organism; and/or
d) calculating the amount of antisense RNA, frequency of 5′ transcript start sites (TSSs), 3′ transcript termination sites (TTSs), splicing pattern, length of poly (A) tail and 3′ polyadenylated sites for the locus.

32. The method of claim 31, further comprising determining the features of RNA products, wherein the features comprise the quality and stability of the RNA products determined by metrics selected from the group consisting of amount/percent of RNA that is full-length and polyadenylated, the size of the region where polyadenylation occurs, the amount of sense vs. antisense RNA, the splicing pattern, the fit to periodicity of the known pattern of RNA degradation occurring at the 3′ ends of the exons, and the length of the poly (A) tail.

33. The method of claim 29, wherein the gene is a transgene and determination of the transgene expression stability leads to prediction of future stability of the expression from the transgene in descendant plants when made homozygous, crossed into different lines, or subjected to post-transcriptional silencing, transcriptional epigenetic silencing or environmental stress.

34. A method of fast-tracking a stable transgenic event, the method comprising selecting a transgenic event that has the most gene-like transgene expression patterns by using the all-in-one RNA-sequencing assay of any one of claims 1-27.

35. The method of claim 34, wherein the gene-like transgene expression patterns comprise accurate transcriptional start sites, patterns of intron splicing, poly (A) tail length and/or clustering of polyadenylation sites.

36. A method of identifying off-type RNAs that trigger RNA decay, RNA degradation, transcriptional or post-transcriptional silencing, the method comprising:
a) sequencing total RNAs or a set of RNAs using the all-in-one RNA-sequencing essay of any one of claims 1-27; and
b) processing the long reads to identify off-type RNAs.

37. A method of diagnosing a disease in an organism, the method comprising:
a) sequencing total RNAs or a set of RNAs from the organism using the all-in-one RNA-sequencing assay of any one of claims 1-27; and
b) comparing the long reads to one or more reference RNA to identify irregularities in the total RNA or the set of RNAs indicative of the presence of a disease in the organism.

38. The method of claim 37, wherein the irregularities comprise RNA degradation, RNA instability, incorrect RNA splicing, incorrect RNA processing, alternative transcriptional start or termination sites, shortening of poly (A) tail length and/or RNA decay.

39. A kit for generating cDNA sequencing libraries using an assay of any one of claims 1-27, the kit comprising adapters comprising unique tags, adapters comprising unique indices, primers for generating cDNAs, primers for amplifying libraries, sequencing adapters and primers, or any combination thereof.
